(19) United States
(12) Patent Application Publication
(10) Pub. No.: US 2010/0174001 A1
(43) Pub. Date: Jul. 8, 2010

(54) ANTIOXIDANT FOR FOOD

(75) Inventors: Joerg Kowalczyk, Eisenberg-Steinborn (DE); Stephan Hausmanns, Wiesbaden (DE); Roland Pahl, Berlin (DE)

Correspondence Address: DICKSTEIN SHAPIRO LLP, 1633 Broadway, NEW YORK, NY, 10019, US

(73) Assignee: SUEDZUCKER AKTIENGESELLSCHAFT MANNHEIM/OCHSENFURT, Mannheim (DE)

(21) Appl. No.: 12/602,231

(22) PCT Filed: May 6, 2008
(86) PCT No.: PCT/EP2008/003612

§ 371 Date: Nov. 30, 2009

(30) Foreign Application Priority Data
Jun. 1, 2007 (DE) 10 2007 026 975.9

(51) Int. Cl. A61K 47/10; C07H 3/04; A61K 8/60; C11B 5/00; A23L 3/34

(52) U.S. Cl. 514/777; 536/120; 426/545; 426/321

(57) ABSTRACT

The present invention relates to the technical field of additives or adjuncts, especially additives that have an antioxidant effect and antioxidants, for food products, animal feed, cosmetics, and pharmaceuticals. The present invention above all provides an improved antioxidant agent for food products, animal feed, cosmetics, and pharmaceuticals and also provides compositions that include this antioxidant agent as preferably the sole supplementary additive that has an antioxidant effect.

The present invention relates to the technical field of additives or adjuncts, especially additives that have an antioxidant effect and antioxidants, for food products, animal feed, cosmetics, and pharmaceuticals. The present invention above all provides an improved antioxidant agent for food products, animal feed, cosmetics, and pharmaceuticals and also provides food products, animal feed, cosmetics, and pharmaceuticals that contain this antioxidant agent as preferably the sole supplementary additive that has an antioxidant effect.

Additives and adjuncts that have an antioxidant effect and that are for food products, pharmaceuticals, and cosmetic agents are known. They primarily suppress the occurrence of degradation products that are created during production or storage of the food product, animal feed, cosmetic, or pharmaceutical when ingredients that are sensitive to oxidation come into contact with atmospheric oxygen or other substances that have an oxidative effect. In the following reference will be made to food products in such a manner that this shall not solely be construed to be preferably food products and animal feed, but also all other agents and compositions, such as cosmetics and pharmaceuticals, that can be used in and on the bodies of animals and humans. As additives to food products, antioxidants enhance the storage stability or aging stability of the food products. These additives may also be a therapeutic and/or prophylactic active ingredient whose effect develops in the animal or human body and suppresses harmful

oxidation processes there. One example of such an antioxidant is ascorbic acid (vitamin C). Ascorbic acid and its salts are added for instance to lemonades, marmalades, condensed milk, and meat products.

Antioxidant agents are food product additives or adjuncts that as a rule are present in a lower concentration compared to an oxidation-sensitive substrate and significantly delay or prevent its oxidation. In addition to the property of themselves being a reducing agent and thus a substrate for oxidative processes, antioxidant agents can be distinguished in that they bind metal ions, especially bivalent iron, that have a catalytic effect in oxidation reactions in the form of chelates and/or suppress radical chain reactions by scavenging the starter radical (scavenging or quenching) or by scavenging an intermediary radical (chain breaking).

Many antioxidants, including ascorbic acid, as well, have the effect of changing the taste of food products, sometimes significantly. This can be used intentionally. Thus ascorbic acid has an acid taste reminiscent of citric acid. As a rule, the antioxidant effect of the antioxidant agent is a function of pH. Thus ascorbic acid is highly antioxidant only in the acid form. However, in many food products the acid taste is not desired or it is not possible to maintain an acid environment, for instance in fresh dairy products. Also, disadvantageous health effects for animals and humans have been discovered in a few known antioxidant agents. These also include ascorbic acid and sulfur dioxide and its salts (sulfite, bisulfite, disulfite, hydrogen sulfite).

Moreover, many of the known antioxidant agents have a sharply reducing effect and therefore under certain conditions lead to undesired reactions with components of the food products.

Many known antioxidant agents, for instance ascorbic acid, are themselves very sensitive to oxidation and/or light, which complicates their technical processability.

There is therefore a need to render accessible for use as antioxidant agents substances that have an antioxidant effect but that do not have the disadvantages of known antioxidant agents.

Another aspect of the invention has to do with brewery technology, especially the production of beer that has long shelf-life and means therefor. Beer is known to be a food product, the taste of which is unstable and subject to a natural aging process. Taste stability is an important quality feature of a beer that is provided for storage. In general the goal is to maintain the original character of the beer from bottling until use. The aging process is primarily characterized by the oxidative degradation of beer ingredients and the so-called “aging components” that result therefrom. They change the taste of the beer in an unfavorable manner. Adding oxygen after fermentation and during bottling is essential for oxidative degradation. Molecular atmospheric oxygen creates reactive oxygen forms, especially the hydroxyl radical. The hydroxyl radical oxidizes especially the following components that are present in beer: ethanol; free fatty acids and isohumulones creating aldehydes and ketones; the

hydroxyl radical also acts as the starter radical for reactions creating other forms of radicals, which also result in aldehydes.

Beer initially includes a number of ingredients that have a reducing effect and that prevent the formation of such disadvantageous oxidation products for a certain period of time. This so-called endogenous antioxidant activity or the endogenous antioxidant potential (EAP) of the beer helps the beer attain a certain storage stability. Ingredients in beer that have an antioxidant effect are especially sulfur dioxide, free phenols, polyphenols, and xanthohumols. If the antioxidant capacity of these ingredients is exhausted, then the shelf life of the beer has been exhausted. Beer therefore has only a limited shelf-life.

Sulfur dioxide is formed in part during main fermentation by the fermentation yeasts that are used. However, the formation of sulfur dioxide depends on the fermentation process and on the strain of yeast used. If the endogenous antioxidant capacity of the beer is to be increased using sulfur dioxide, a special fermentation process and a special yeast must be selected; this limits the flexibility that can be enjoyed when practicing the art of brewing.

Different technological measures are used during beer production in order to increase the proportion of such phenolic substances in the beer. Some of these methods are complex and change the brewing outcome; in addition, the flexibility that can be enjoyed when practicing the art of brewing is limited.

One known antioxidant agent added to beer is sulfur dioxide. It is generally added to those beers that are provided for lengthy storage, for instance for

export overseas. Sulfur dioxide changes the taste of the beer for the worse from the outset. Other known oxidation agents such as ascorbic acid also change the taste of the beer in an unfavorable manner.

It is therefore the object of the present invention to provide means for us to increase the endogenous antioxidant capacity (EAP) of beer, mixed beer beverages, and other brewery products without having to accept disadvantages in taste and other disadvantages associated with known antioxidant agents.

The technical problem underlying the invention is solved by providing an antioxidant agent or an antioxidant agent composition preferably for use in food products, animal feed, cosmetics, or pharmaceuticals, which agent contains isomaltulose (palatinose) as a component having an antioxidant effect. The inventive antioxidant agent preferably includes isomaltulose as the sole component having an antioxidant effect or comprises it. In one variant of the invention, isomaltulose is a component of an antioxidant agent composition that, in addition to isomaltulose, contains at least one additional component that synergistically supports the antioxidant effect of isomaltulose. Such a synergistic effect is preferably associated with the complexing or chelating of metal ions that have an oxidizing, catalyzing effect.

The inventors surprisingly found that the addition of isomaltulose to food products significantly enhances their oxidative stability. Isomaltulose is an effective adjunct in food products for enhancing

storage stability, aging stability, and oxidation stability. Isomaltulose prevents or effectively reduces the occurrence of oxidation degradation products, so-called aging components, that limit shelf life for instance in food products such as beer or beer-like beverages by having a negative effect on taste and/or due to an unhealthy effect. Isomaltulose has a protective action for oxidation-sensitive food product components such as dyes, flavorings and pharmaceutical active substances, unsaturated fatty acids, including especially omega-3 and/or omega-6 fatty acids and comparable fatty acids. Without having been attached to the theory, isomaltulose surprisingly indicates clearly greater and thus also more effective antioxidant effect compared to other known reducing sugars such as glucose.

Thus, the invention provides for using isomaltulose, a substance already known in the food products industry, as an antioxidant agent preferably for food products, cosmetic products, and pharmaceutical preparations. Isomaltulose is preferably used as the sole antioxidant agent added to the product.

Isomaltulose is a disaccharide that, as is known, can be used like sucrose. Isomaltulose is currently primarily used as a substitute for sucrose in known food formulations that contain sucrose. Isomaltulose is used as a body-creating sweetener (“bulk substance”). In contrast, the present invention provides a different teaching: isomaltulose is used as a food adjunct or additive that has an antioxidant effect. The invention is thus in a different technical area of application; in accordance with the invention,

isomaltulose can also be used in compositions and food products in which the sweetening effect of isomaltulose and/or its function as a body-creating component is not necessary and/or is not used. The inventive use of isomaltulose thus lies outside of the area of sweets and/or outside of food products that contain carbohydrates. These include for instance food products that are rich in protein and/or fat such as dairy products (cheese, yogurt, etc.) and preparations that contain oil or fat (margarine, edible oils, etc.).

The invention also relates to food products, cosmetic products, and pharmaceutical preparations that include the inventive antioxidant agent and relates to the use of isomaltulose as an antioxidant agent in such products. The product preferably includes only isomaltulose as the single antioxidant agent added to the product. This does not preclude at least one other component being added in addition to the isomaltulose, which component preferably supports or enhances the antioxidant effect of isomaltulose in a synergistic manner.

An inventive food product that contains isomaltulose as an antioxidant agent is preferably selected from:
i. Dairy products and milk products such as cheese, butter, yogurt, kefir, curd cheese, sour cream, buttermilk, cream, condensed milk, powdered milk, whey, lactose, milk protein, mixed dairy, reduced fat milk, mixed whey, and milk fat products and preparations;
ii. Puddings, cremes, mousses, and other desserts;
iii. Milk fat products, mixed fat products, edible fats, edible oils;
iv. Bakery products such as bread, including cookies and fine baked goods, long-life bakery products, cracker products, and waffles;
v. Spreads, especially spreads that contain fat, margarine products, and shortening;
vi. Instant products and broth products;
vii. Fruit products and preparations such as jams, marmalades, jellies, fruit preserves, fruit pulps, fruit purees, fruit juices, fruit juice concentrates, fruit nectar, and fruit powder;
viii. Cereals, muesli, and cereal mixtures, as well as prepared products that contain cereals such as muesli bars and breakfast products;
ix. Primarily non-alcoholic beverages, bases for beverages and drink powders, cocoa beverages, cocoa beverage powders;
x. Primarily alcoholic beverages and fermentation products, wine, mixed wine beverages, beer, mixed beer beverages, alcohol-free beer and mixed beer beverages, reduced alcohol beer and mixed beer beverages;
xi. Meat and sausage products;
xii. Sweets such as chocolates, hard caramels, soft caramels, chewing gum, sugar candies, fondant products, jelly products, licorice, marshmallow candies, flakes, sugar candies, candy tablets, candied fruits, brittles, nougat products, “ice chocolate”, marzipan, ice cream.

Naturally the invention also has to do with food products that are derived from the aforesaid food products, especially special dietetic food products. The invention furthermore also relates to food products that are not or are not exclusively intended or suitable for human consumption; these include animal feed, pet food, premixes for pet food, high starch animal feed, high protein animal feed, high fat animal feed, pellets, and concentrated feed.

The target of the inventive use of isomaltulose as an antioxidant agent is the food products that generally include one component that is sensitive to oxidation and subject to aging processes, which would reduce the shelf-life of the food product. Among these are inventive substances or substance mixtures that are subjected to oxidative degradation, especially during production and/or storage of the food product. This oxidative degradation is preferably triggered by contact with components that contain oxygen, especially by contact with atmospheric oxygen. The oxidative degradation can also be caused by other substances that are included in the food product or in a food product composition and that themselves have an oxidative effect. These include for instance oxidizing acids, metals that have a high oxidation state and their

compounds, preservatives that have an oxidizing effect, and other oxygen, sulfur, or halogen compounds that have an oxidizing effect. In such food products, isomaltulose as an antioxidant agent also enhances stability with respect to free radicals and suppresses the formation of free radicals.

One preferred subject-matter of the invention is a food product that includes isomaltulose as an antioxidant agent, preferably the only antioxidant agent, and is a dairy product or mixed dairy product, especially yogurt, and includes unsaturated fatty acid, especially omega-3 fatty acid, omega-6 fatty acid, or the like, as a component that is sensitive to oxidation. Surprisingly it has been demonstrated that as an antioxidant agent isomaltulose effectively suppresses the oxidative degradation of omega-3 fatty acid and omega-6 fatty acid. In a dairy product that contains isomaltulose, especially yogurt that contains isomaltulose, added omega-3 fatty acid, omega-6 fatty acid, and another component that is sensitive to oxidation (see above) is only mildly degraded, even given lengthy storage.

Another preferred subject-matter of the invention is the food product beer or modified forms of it such as mixed beer beverages or reduced alcohol or alcohol-free beer or mixed beer beverages that include isomaltulose preferably as the sole antioxidant agent, particularly preferred as the sole added antioxidant agent. Surprisingly, it has been demonstrated that a beer that contains isomaltulose has particularly good storage stability. Even given lengthy storage, unfavorable changes in taste that are caused by aging are kept within tolerable limits. Isomaltulose stabilizes components in the beer that are sensitive to oxidation and increases the EAP value. Isomaltulose prevents premature enrichment of the aging

components that are responsible for the so-called “aging taste” of stored beer. Among the aging components in beer that can be effectively suppressed by the presence of isomaltulose are 2-methylbutanal, 2-furfural, E-2-nonenal, nicotinic acid ethyl ester, and gamma-nonalactone. This includes especially E-2-nonenal, which is frequently known as a “marker” for beer aging. After 14 days of storage, its portion in a beer that contains isomaltulose decreases by up to 20% compared to a control beer.

In general the oxidative stability of a sample, for instance of a food product, can be determined by means of ESR spectroscopy (electron spin resonance spectroscopy). This method, which was primarily established for determining the aging stability of beer, can also be used with other samples that are sensitive to oxidation. ESR spectroscopy determines the endogenous antioxidant potential (EAP value) of the sample. This is used for instance in order to be able to gain some knowledge about the storage stability that can be expected for beer. The method is based on accelerated aging (forced aging test) of the sample at elevated temperatures (generally 60° C. to 63° C.). Spectroscopic detection of radical scavenger adducts is used to determine the so-called “lag time” for the sample. ESR spectroscopy is an indirect indication of radical generation in the sample during the course of accelerated sample aging. The radicals that occur in the sample are very reactive and in aqueous solutions generally have only a very short life. A radical scavenger, a so-called “spin trap”, that can absorb diffusible radicals is used. The stable radical adducts that result can be detected by their spectrographic characteristics using ESR

spectroscopy. For a certain period of time, the so-called “lag phase”, the sample is able to prevent or delay radical formation due to its endogenous antioxidant potential (EAP). If the antioxidant potential of the sample has been exhausted, radical generation proceeds unimpeded. This point in time is extrapolated as the EAP value. What follows then is a rapid increase in the signal intensity in the ESR spectrum, which results from the now cumulatively generated “spin trap” adducts.

One important criterion when determining the aging stability of food products, especially beverages, is the so-called BAX value. The higher it is, the better. Adding isomaltulose to beverages, in particular to beer, increases the BAX value of the beverage.

The reduction potential of a beverage, in particular beer, is found according to the MEBAK (Central European Brewery Analysis Commission) in order to determine the aging stability of food products, especially beer. Adding isomaltulose to the beer increases the reducing potential of the beer.

One subject-matter of the invention is consequently also the use of isomaltulose for enhancing aging stability, oxidation stability, and/or storage stability of food products, animal feeds, cosmetics, and pharmaceuticals, especially of food products, animal feed, cosmetics, and pharmaceuticals that are sensitive to oxidation, especially beer, mixed beer beverages, instant beverages, and instant cocoa beverages; especially the use of isomaltulose for reducing the occurrence of aging components in beer or mixed beer beverages, which components have a negative effect on taste. Finally, one

subject-matter of the invention is also the use of isomaltulose for reducing the oxidation of oxidation-sensitive dyes, flavorings, pharmaceutical active ingredients, and/or unsaturated fatty acids, especially omega-3 fatty acids and the like in food products, especially in yogurt and the like.

The present invention shall be illustrated in greater detail using the following examples and figures, but this shall not be construed as limiting.

### EXAMPLE 1

### Stabilizing Omega-3 Fatty Acids in Dairy Products

The ability of isomaltulose to suppress or reduce the oxidative degradation of omega-3 fatty acids that are embedded in a yogurt matrix was tested.

Whole-milk yogurt (yogurt, mild, 3.5% fat; Milram) was used, and DHA CL (Lonza) was stirred in as omega-3 fatty acid. The following yogurt preparations were produced:

Batch 1 (in accordance with invention): 5 g isomaltulose per 100 g
Batch 2 (in accordance with invention): 10 g isomaltulose per 100 g
Batch 3: 5 g fructose per 100 g
Batch 4: 10 g fructose per 100 g
Batch 5: 5 g sucrose per 100 g
Batch 6: 10 g sucrose per 100 g.

150 mg DHA-CL (Lonza) and about 40 mg of a saturated fatty acid (C22:0) were each stirred by means of an Ultraturrax into 240 g of each of these batches as an internal standard. All actions were performed under a protective nitrogen atmosphere.

Immediately after the yogurt preparations were produced, blank samples were drawn and the recovery rate of the omega-3 fatty acid in the just-produced preparations was determined.

For measuring the omega-3 fatty acids, 0.85 mL of each yogurt preparation were pipetted into a test-tube (Eppendorf), weighed, and 1 mL tert-butyl-methylether was added to each; this was followed by vigorous shaking. After shaking for about 3 minutes, the test-tubes were centrifuged for 3 min at 13,000 rpm. Then 200 μl of the clear supernatant were removed from each and 100 μl THMS were added. 1 μl of this solution was injected into a gas chromatograph (Agilent, type 6890) that was optimized in a manner known per se for detecting the fatty acids used.

The recovery rate in all of the blank sample batches was uniformly from 97 to 99% of the originally used omega-3 fatty acid.

The yogurt preparations produced were then stored for 11 days at 5° C. A certain tendency for separation was observed. Prior to analysis, the preparations were therefore vigorously shaken again, so that the separation was eliminated macroscopically. Each of the samples

was analyzed using gas chromatography as described in the foregoing for the blank samples.

Table 1 provides the recovery rates for the omega-3 fatty acids in each of the yogurt preparations after a storage period of 11 days. Surprisingly, the preparations that contain isomaltulose (batches 1 and 2) have the highest recovery rates; the recovery rates found were 95 to 97% of the originally used omega-3 fatty acids. The control samples with the added fructose or sucrose had significantly lower recovery rates.

TABLE 1

| | | | Original DHA | DHA |
|---|---|---|---|---|
| | Portion | Molarity | Value | Recovery |
| Sugar | [g/100 g] | [mmol/100 g] | [mg/100 g] | [%] |
| Isomaltulose | 5 | 13.8 | 26.6 | 97 |
| (According to | 10 | 27.8 | 24.1 | 95 |
| the invention) | | | | |
| Fructose | 5 | 27.7 | 26.8 | 88 |
| (Reference) | 10 | 55.5 | 24.4 | 91 |
| Sucrose | 5 | 14.6 | 24.9 | 75 |
| (Reference) | 10 | 29.2 | 23.2 | 78 |

Continued storage of the yogurt preparations for a number of weeks confirms this effect of the isomaltulose that was found in the first 11 days of storage—suppression of the oxidative degradation of omega-3 fatty acids.

Surprisingly, it has been demonstrated that, even at higher molarity, fructose, which acts as a reducing sugar via the keto-en(di)ol tautomery,

has a significantly lower protective effect than isomaltulose. The surprisingly strong antioxidant effect of isomaltulose cannot be attributed solely to the presence of reactive aldehyde groups. The reducing activity (redox potential) of aldehyde sugars was generally considered to be too low for the strong antioxidant effect found to have been derived solely therefrom.

### EXAMPLE 2

### Stabilizing Instant Beverages

The endogenous antioxidant potential (EAP), and thus the storage stability, of instant beverages having components sensitive to oxidation was investigated. For this, instant beverages from instant beverage recipes described in the following were prepared fresh, each of them including isomaltulose or sucrose as the base.

The following instant powder batches were produced (figures provided in wt. %):

Batch 1: “Orange drink” (according to invention)

| | |
|---|---|
| Isomaltulose | 94.09 |
| Citric acid (anhydrous) | 4.97 |
| Trisodium citrate (Merck) | 0.26 |
| Tricalcium phosphate (Merck) | 0.22 |
| Dye E102 (85%) | 0.01 |
| Dye E110 (85%) | 0.016 |
| Sodium carboxymethyl cellulose, E466 | 0.10 |
| Orange juice aroma (No. 655228, Symrise) | 0.064 |
| Orange aroma (No. 614756, Symrise) | 0.24 |
| Sucralose (Splenda) | 0.03 |

Batch 2: “Orange drink” (reference example)

| | |
|---|---|
| Sucrose | 94.12 |
| Citric acid (anhydrous) | 4.97 |
| Trisodium citrate (Merck) | 0.26 |
| Tricalcium phosphate (Merck) | 0.22 |
| Dye E102 (85%) | 0.01 |
| Dye E110 (85%) | 0.016 |
| Sodium carboxymethyl cellulose, E466 | 0.10 |
| Orange juice aroma (No. 655228, Symrise) | 0.064 |
| Orange aroma (No. 614756, Symrise) | 0.24 |

Batch 3: “Chocolate drink” (according to invention)

| | |
|---|---|
| Isomaltulose, ground (Palatinose TM-PA) | 79.12 |
| Cocoa powder, very low fat, dark GT 150 (Gerkens) | 20.00 |
| Lecithin Metarin P IP (Cargill) | 0.75 |
| NaCl (No. 71383, Fluka) | 0.05 |
| Vanillin (No. 130879, Symrise) | 0.05 |
| Sucralose (Splenda) | 0.03 |

Batch 4: “Chocolate drink” (reference example)

| | |
|---|---|
| Sucrose | 79.15 |
| Cocoa powder, very low fat, dark GT 150 (Gerkens) | 20.00 |
| Lecithin Metarin P IP (Cargill) | 0.75 |
| NaCl (No. 71383, Fluka) | 0.05 |
| Vanillin (No. 130879, Symrise) | 0.05 |

The batches were each used like beverage powders and dissolved in water. Approx. 7 to 20 g beverage powder were used per 200 mL finished instant beverage.

Then the batches were subjected to accelerated aging (forced aging test) at 63° C. During the accelerated aging, the antioxidant potential (EAP value) was determined with ESR spectroscopy in a manner known per se using special spin-trap reagents.

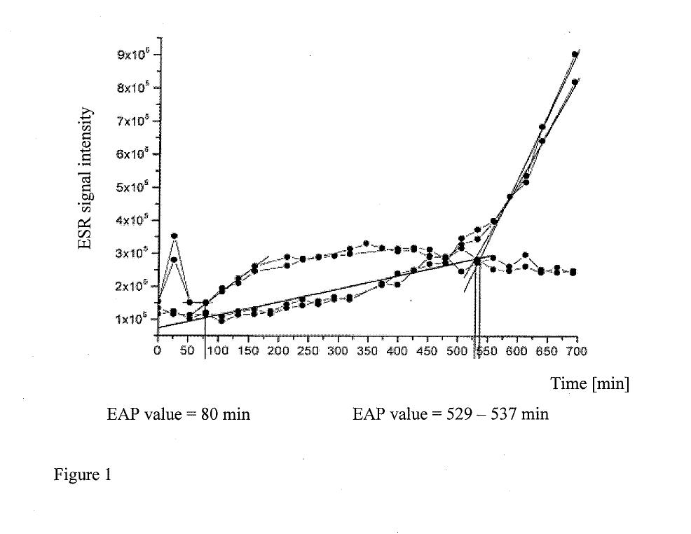

FIG. 1 depicts the measured curves from the ESR spectroscopy for the “chocolate drink” instant beverage.

Table 2 lists the measurement series for the “chocolate drink” beverage from the different test batches and measurement runs.

TABLE 2
EAP Values for Instant Beverage [min]

| Batch 3 | |
|---|---|
| (According to | Batch 4 |
| the invention) | (Reference) |
| 504-510 | <80 |
| 529-537 | <80 |

Isomaltulose made an extraordinary enhancement in the oxidative stability of the instant beverage produced.

A generally higher EAP value was found in the instant “orange drink” beverages from batches 1 and 2 (approx. 800 min or more). Inventive batch 1 having isomaltulose again exhibits elevated EAP values compared to the reference batch 2.

### EXAMPLE 3

### Beer with Enhanced Taste Stability

For testing the influence on taste stability of beer, isomaltulose was added to a commercial light beer (containing almost no carbohydrates) and it and a reference sample were subjected to defined beer aging. The following analyses were performed: comparison tasting, measurement of the reducing power of the beer, measurement of the oxidation stability by means of ESR, and gas chromatography determination of the aging components.

In a first test batch, in accordance with the invention approximately 2 g/100 mL isomaltulose were added to a commercial light beer. The commercial beer, without isomaltulose, was used for the reference batch (batch 2).

Batch 1: Commercial light beer with isomaltulose
added (according to invention)

| | |
|---|---|
| Original wort [gem %] | 10.97 |
| Residual extract, apparent [wt. %] | 1.85 |
| Residual extract, actual [wt. %] | 3.55 |
| Alcohol content [vol. %] | 4.66 |
| Bitter units [BE] | 23 |

Batch 2: Commercial light beer without isomaltulose
added (reference example)

| | |
|---|---|
| Original wort [gem %] | 9.1 |
| Residual extract, apparent [wt. %] | 0 |
| Residual extract, actual [wt. %] | 1.66 |
| Alcohol content [vol. %] | 4.78 |
| Bitter units [BE] | 24 |

All batches were stored at 28° C. for 2 weeks.

### Tasting Results

Table 3 provides the tasting results after two weeks' storage at 28° C. (n=10 tasters, ideal value=5)

TABLE 3

| | Batch 1 | |
|---|---|---|
| | (According to | Batch 2 |
| Parameter | invention) | (Reference) |
| Smell | 4.91 | 4.89 |
| Purity of taste | 4.86 | 4.12 |
| Full body | 4.98 | 4.3 |
| Sparkle | 4.52 | 4.6 |
| Bitter quality | 4.11 | 3.84 |

The stored beer with the added isomaltulose was judged to be better during the tasting. There was almost no difference in terms of the smell, but there were significant differences in the quality of the taste, the body, and the bitter quality.

### Measurement of Reducing Potential

Using the MEBAK method, the reducing potential of the beers was measured using spectral photometry by means of a tannometer in a manner known per se. The reduction of 2.6-dichloroindophenol was tracked over 1 min.

TABLE 4

| | Batch 1 | |
|---|---|---|
| | (According to | Batch 2 |
| Parameter | invention) | (Reference) |
| Reducing power | 51 | 45 |

Table 4 provides the results (average of two measurements). Adding isomaltulose causes the reducing power to increase by 6 points. According to MEBAK, a value greater than 60 (dimensionless number) is very good for light beer, and a value less than 45 is poor. Isomaltulose raises the reducing potential of the commercial light beer used so that it moves up and out of the poor classification. Thus adding isomaltulose causes an immediate enhancement in the measurable quality of beer.

### Determination of Aging Components

Certain chemical substances are associated with the aging taste in stored beer. The substances responsible for the aging taste (“aging components”) should be found.

Both batches were stored at 26° C. for 14 days. Then the aging components were detected and quantified in a manner known per se using chromatography.

Table 5 provides the aging components found by chromatography after 14 days' storage at 26° C.

TABLE 5

| | Batch 1 | | |
|---|---|---|---|
| Aging components | (According to | Batch 2 | Difference |
| [μg/L] | invention) | (Reference) | [%] |
| 3-Methylbutanal | 8.5 | 8.5 | 0.0 |
| 2-Methylbutanal | 2.9 | 3.4 | −17.2 |
| 2-Furfural | 250.6 | 294.2 | −17.4 |
| Heptanal | n.d. | n.d | 0.0 |
| Methional | n.d. | n.d. | 0.0 |
| Benzaldehyde | 3.2 | 3.1 | 3.1 |
| Octanal | n.d. | n.d. | 0.0 |
| Phenylethanal | 19.2 | 18.8 | 2.1 |
| E-2-nonenal | 3.6 | 3.9 | −8.3 |
| Nicotinic acid ethyl ester | 51.9 | 60.7 | −17.0 |
| Gamma-nonalactone | 57.4 | 62.7 | −9.2 |

3-Methylbutanal remained unchanged, heptanal and methional were not found in detectable quantities in either sample and therefore there are no changes in these values, either. In the presence of isomaltulose, the majority of the analyzed substances were formed significantly less, including trans-2-nonenal (E-2-nonenal), which is considered a crucial aging parameter in much research; the reduction in the aging components ranged from 9 to 17%.

### Measurement of Oxidative Stability

Another analysis for determining taste stability of beer that is generally recognized as meaningful is the measurement by means of ESR (electron spin resonance spectroscopy) of the oxidative stability of the sample. The BAX value is determined according

to the methodology of Methner and Kunz. According to Methner and Kunz (Methner and Kunz, 2006 Precise Prognoses of Oxidative Beer Stability by means of ESR Spectroscopy, Brauerei Forum 2006: 7-9), the BAX value is measured when determining the oxidative stability of beer by means of ESR. BAX (Beverage Antioxidant Index) is calculated as follows: BAX=ΔEAP value/ΔSO₂ content [min l/mg].

The linear association between SO₂ content and signal intensity of the ESR measurement (EAP value) is a function of intrinsic beer-specific factors, which are different for each beer, and thus the curves have different slopes. During the measurement, the samples are doped with different SO₂ contents and the BAX value is calculated from the resultant curves; the higher the BAX value the better. Moreover, the highest absolute signal intensity is used for the assessment, since it is a measure of the radical substances formed by oxidative processes during the measurement.

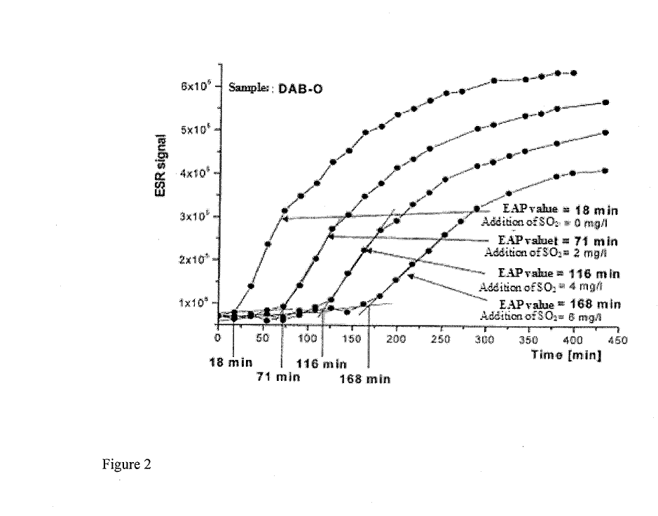

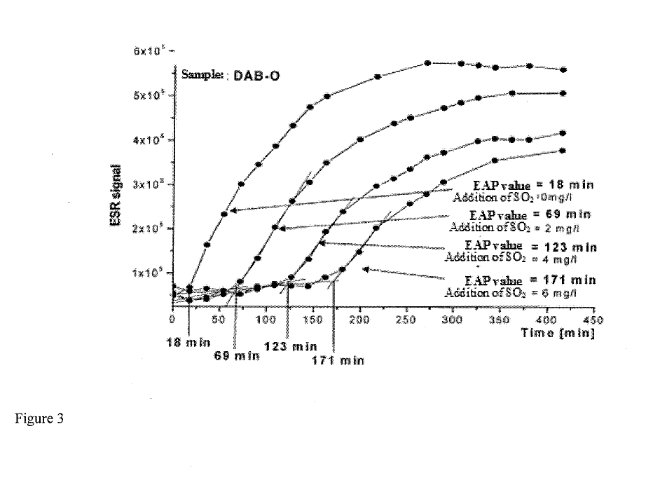

FIGS. 2 and 3 display the curves recorded during the spectroscopic EAP measurement. The BAX values shown in Table 6 were calculated from the curves.

FIG. 2 depicts the curve progressions for light beer without isomaltulose in the BAX measurement (batch 2, reference batch).

FIG. 3 depicts the curve progressions for light beer with isomaltulose in the BAX measurement (batch 1, according to invention).

TABLE 6

| | Batch 1 | |
|---|---|---|
| | (According to | Batch 2 |
| Parameter | invention) | (Reference) |
| BAX value [min/mg SO₂] | 25.7 | 24.8 |

The inventively attained BAX value is better than that for the reference sample with no isomaltulose. The comparison of the absolute maximum values for the measured signal intensity in all of the test series provided higher final values for the batches with no isomaltulose. This means that overall fewer radical oxidation products result in beer in the presence of isomaltulose than in the reference sample. FIG. 3 provides the results.

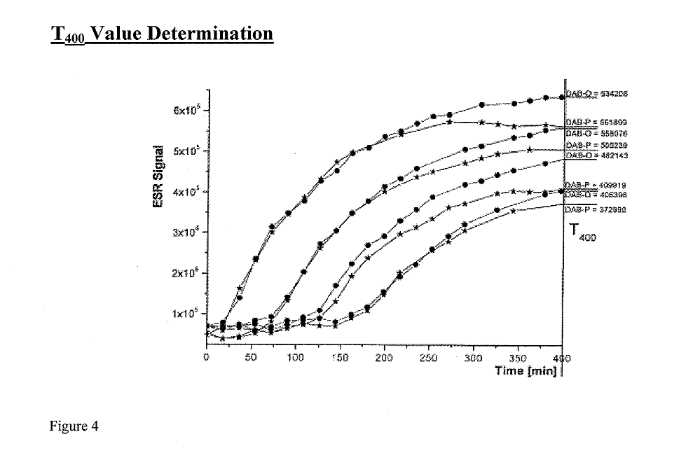

FIG. 4 provides a comparison of the signal intensities for the beers, with and without isomaltulose.

### SUMMARY OF RESULTS

Adding isomaltulose has a positive effect on the storage stability and taste stability of the beer. Adding 2 g/L is considered relatively little; higher portions boost the effects.

1. (canceled)

2. In a product which is a food product, animal feed, cosmetic, or pharmaceutical containing an antioxidant agent, the improvement which comprises the antioxidant comprising isomaltulose.

3. A product in accordance with claim 2 in which the isomaltulose is the sole antioxidant.

4. Product in accordance with claim that includes at least one unsaturated fatty acid that is sensitive to oxidation.

5. Product in accordance with claim 4, in which the unsaturated fatty acid is an omega-3 or omega-6 fatty acid.

6. Product in accordance with claim 2 which is a food product selected from the group consisting of dairy or milk product, desserts, edible fats and oils, bakery products, spreads and shortening, instant and broth products, fruit products, cereals and muesli, non-alcoholic beverages and bases or powders therefor, alcoholic beverages and fermentation products, meat and sausage products, and sweets.

7. Product in accordance with claim 2 that is an animal feed selected from the group consisting of pet food, premix for pet food, high starch animal feed, high protein animal feed, high fat animal feed, pellets, or concentrated feed.

8. Product in accordance with claim 2 that is a dairy, yogurt, or mixed dairy product that contains omega-3 or omega-6 fatty acid.

9. Product in accordance with claim 2 that is a bier, mixed beer beverage, or alcohol-free or reduced alcohol beer or mixed beer product.

10. A method of enhancing the aging or storage stability, or both, of food products, animal feed, cosmetics, or pharmaceuticals by incorporating an enhancing agent therein in which the enhancing agent is isomaltulose.

11. A method of enhancing the oxidation stability of food products, animal feed, cosmetics, or pharmaceuticals by incorporating an antioxidant therein in which the antioxidant is isomaltulose.

12. A method of reducing the occurrence of aging components in food products, animal feed, cosmetics, or pharmaceuticals, which components have a negative effect on taste, by incorporating an anti-aging agent therein in which the anti-aging agent is isomaltulose.

13. A method according to claim 11 in which the food products, animal feed, cosmetics, or pharmaceuticals contain an unsaturated fatty acid.

14. (canceled)

15. A method in accordance with claim 13, in which the unsaturated fatty acid is an omega-3 or omega-6 fatty acid.

16. A method in accordance with claim 13 in which the isomaltulose is the sole antioxidant.

17. A method in accordance with claim 12 in which the isomaltulose is the sole anti-aging agent.

18. A method in accordance with claim 11 in which the isomaltulose is the sole antioxidant.
